(19) United States
(12) Patent Application Publication
(10) Pub. No.: US 2006/0100518 A1
(43) Pub. Date: May 11, 2006

(54) Automated diastolic function analysis with ultrasound

(76) Inventor: Sriram Krishnan, Exton, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/179,422

(22) Filed: Jul. 12, 2005

Related U.S. Application Data
(60) Provisional application No. 60/620,761, filed on Oct. 21, 2004.

(51) Int. Cl. A61B 8/02

(52) U.S. Cl. 600/450

(57) ABSTRACT

Automated analysis of heart function is provided with ultrasound images. A clip of ultrasound images of the heart is used to detect two or three dimensional motion, such as determining a two-dimensional motion vector by tracking wall segments with B-mode data. The amplitude of motion during the e-wave and/or a-wave phase of diastole is extracted for diagnosis assistance.

### RELATED APPLICATIONS

The present patent document claims the benefit of the filing date under 35 U.S.C. §119(e) of Provisional U.S. Patent Application Ser. No. 60/620,761, filed Oct. 21, 2004, the disclosure of which is hereby incorporated by reference.

### BACKGROUND

The present invention relates to detection of medical abnormalities. In particular, diastolic function analysis is performed with ultrasound data.

Regional diastolic function assessment is an important indicator of early heart failure. Currently, cardiac wall motion is analyzed to detect abnormalities during systole (the contraction phase of the heart). For example, echocardiography (e.g., stress echo) includes segmented wall motion analysis. The left ventricle wall is divided into a plurality of segments (e.g., 16 or 17) according to a standard recommended by the American Society of Echocardiography (ASE). Various standardized ultrasound views are obtained to acquire image data information for each left ventricular segment. The views are standardized such that the segments are roughly in line with a presumed distribution of three major coronary artery segments. The echocardiographer visually inspects the acquired image data to access global function and regional abnormalities. Based on the cardiographer's assessment, a wall motion score is assigned to each segment in accordance with the ASE scoring scheme. The absolute and relative systolic excursion and timing of excursion is assessed to provide a report of negative (non-pathological) or positive (pathological) findings. Such wall motion diagnosis may require significant training and experience on the part of the echocardiographer. However, cardiac wall motion can also be used to assess the heart during diastole (expansion phase of the heart).

There are four classic phases of diastole: the isovolumetric relaxation phase, early wave during rapid filling phase (E wave), diastasis phase, and late wave filling phase (A wave). Currently, diastolic function analysis is done, with ultrasound, using a pulsed-wave Doppler technique. Blood velocity is measured at various locations in the left ventricle. However, PW Doppler does not directly interrogate the various specific segments of the myocardium.

Another approach to diastolic analysis in ultrasound is to identify the velocity of tissue or tissue motion with Doppler processes. Tissue motion may indicate useful information during the four phases of diastole. Tissue motion can be used instead of PW Doppler blood flow velocity measurements, and the early wave and late wave filling can be visualized. However, Doppler tissue motion, like Doppler blood flow velocity, only measures the velocity towards or away from the transducer.

Automated border detection and motion techniques may be used to assess diastolic function. Volume and filling rates are assessed, but without measuring tissue velocities.

### BRIEF SUMMARY

By way of introduction, the preferred embodiments described below include methods, systems and instructions on computer readable media for automated analysis of heart function with ultrasound. A clip of ultrasound images of the heart is used to detect two or three dimensional motion, such as determining a two-dimensional motion vector by tracking wall segments with B-mode data. The amplitude of motion during the e-wave and/or a-wave phase of diastole is extracted for diagnosis assistance.

In a first aspect, a method is provided for automated analysis of heart function with ultrasound. Ultrasound data representing a heart at different times is acquired. Two or three dimensional motion is determined with a processor for a plurality of locations from the ultrasound data. An amplitude of the two or three dimensional motion during a rapid filling period, an atrial contraction or both the rapid filling period and the atrial contraction is extracted.

In a second aspect, a computer readable storage medium has stored therein data representing instructions executable by a programmed processor for automated analysis of heart function with ultrasound. The instructions are for: acquiring ultrasound data representing a heart at different times; determining two or three dimensional motion for each of a plurality of segments of the heart from the ultrasound data; and identifying amplitudes of each of the two or three dimensional motions during an e-wave period, an a-wave period or both the e-wave and a-wave periods.

In a third aspect, a system is provided for automated analysis of heart function with ultrasound. A memory is operable to store ultrasound data representing a myocardium at different times. A processor is operable to determine multi-dimensional motion for each of a plurality of locations from the ultrasound data and operable to determine amplitudes of the multi-dimensional motions of the locations during a rapid filling period, an atrial contraction or both the rapid filling period and the atrial contraction. A display is operable to display information as a function of the amplitudes.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for automated analysis of heart function;

FIG. 2 is a flow chart diagram of one embodiment of a method for automated analysis of diastolic heart function; and

FIG. 3 is one embodiment of a display of diastolic parameters.

### DETAILED DESCRIPTION OF THE DRAWINGS AND PRESENTLY PREFERRED EMBODIMENTS

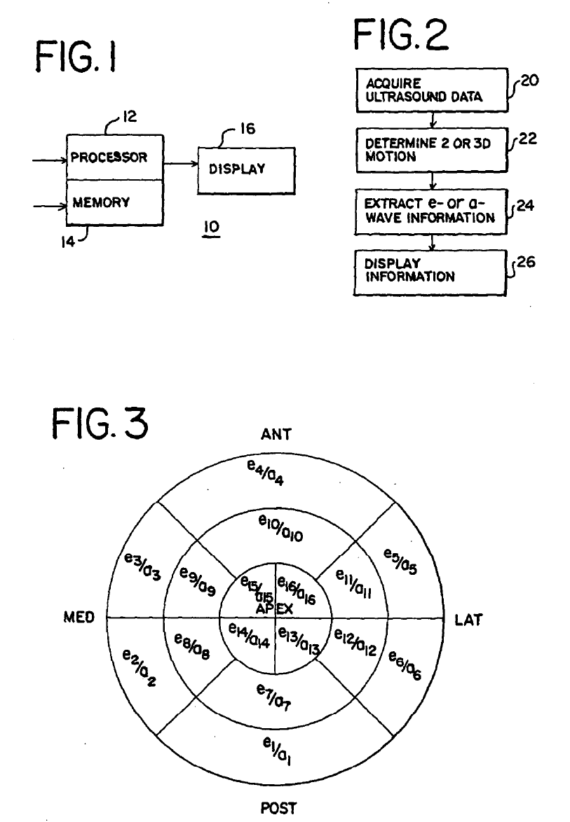

FIG. 1 shows a system 10 for automated analysis of heart function with ultrasound. The system 10 includes a processor 12, a memory 14 and a display 16. Additional, different or fewer components may be provided. The system 10 is a personal computer, workstation, medical diagnostic imaging system, network, or other now known or later developed system for automatically determining motion and extracting motion amplitude for diastolic analysis with a processor. For example, the system 10 is a computer aided diagnosis system. Automated assistance is provided to a physician for identifying abnormal or normal operation of a heart or myocardium. The automated assistance is provided after subscription to a third party service, purchase of the system 10, purchase of software or payment of a usage fee.

The processor 12 is a general processor, digital signal processor, application specific integrated circuit, field programmable gate array, analog circuit, digital circuit, combinations thereof or other now known or later developed processor. Any of various processing strategies may be used, such as multi-processing, multi-tasking, parallel processing or the like. The processor 12 is responsive to instructions stored as part of software, hardware, integrated circuits, film-ware, micro-code and the like.

In one embodiment, the processor 12 is a classifier programmed with thresholds, filters or other learned, predetermined or trained parameters. For example, the processor 12 is a classifier, such as a model or trained classification system. Recommendations or other procedures provided by a medical institution, association, society or other group are reduced to a set of computer instructions. In response to ultrasound images, the classifier implements the recommended procedure for classifying, scoring or identifying normal or abnormal states. In other embodiments, the classifier is implemented with machine learning techniques, such as training a neural network using sets of training data obtained from a database of patient cases with known diagnosis. The learning may be an ongoing process or be used to program a filter or other structure implemented by the processor 12 for later existing cases. Any now known or later developed classification schemes may be used, such as cluster analysis, data association, density modeling, probability based model, a graphical model, a boosting base model, a decision tree, a neural network, filtering or combinations thereof.

The classifier includes a knowledge base for analyzing ultrasound images for diastolic function. The knowledge base is learned, such as parameters from machine training, or programmed based on studies or research. The knowledge base may be disease, institution, or user specific, such as including procedures or guidelines implemented by a specific hospital. The knowledge base may include parameters or software defining a learned model.

The processor 12 determines multi-dimensional motion for each of a plurality of locations from ultrasound data. The ultrasound data is from a processing path before imaging, such as being pre-detected data, B-mode detected data, pre-scan converted data or scan converted data, or is image data, such as RGB values. The ultrasound data represents one or more two dimensional regions, such as associated with different views of the heart (e.g., A4C, A2C, PSAX, PLAX and/or ALAX). Alternatively, the ultrasound data corresponds to a two dimensional representation of a volume (i.e., rendered three-dimensional image) or corresponds to a volume (i.e., a three dimensional data set).

The ultrasound data represents acoustic echoes from the heart tissue and/or fluid. For example, the ultrasound data represents, in part, the myocardial wall of the heart. The ultrasound data may be grouped to represent different myocardial wall segments, such as the ASE standard 16 segments or other segment groupings. Each segment is associated with a line or two-dimensional patch.

The motion is determined using correlation, filtering, minimum sum of absolute differences, border detection, classification, speckle tracking or other now know or later developed techniques. Multiple images or portions of images representing the same or similar view of the heart are compared. The same locations are identified in the multiple images. Using B-mode data, a motion vector in two or three dimensions is identified from sequential images. For example, the tracking disclosed in U.S. Pat. No. ______ (Published Application No. 2004/0208341), the disclosure of which is incorporated herein by reference, is used. One or more control points along an initial contour of a global shape are defined. Each of the one or more control points is tracked as the object is in motion. Uncertainty of a location of a control point in motion is represented using a number of techniques, such as covariance matrix. The uncertainty to constrain the global shape is exploited using a prior shape model. In an alternative embodiment, multiple appearance models are built for each control point and the motion vectors produced by each model are combined in order to track the shape of the object.

As an example using pre-detected data (e.g., in-phase and quadrature or radio frequency ultrasound data), a velocity is determined by correlation. Where the pre-detected data representing a same location is acquired closely in time, the correlation indicates velocity. The magnitude of the motion is provided by the velocity. The angle of the motion is the direction where the correlation is maximized. Other techniques, such as pattern matching, may be used to determine motion associated with a feature, point, line, area or spatial location.

The processor 12 determines the amplitudes (e.g., velocity) of the multi-dimensional motions of the locations. Since the amplitudes are from two or three dimensional motion vectors, the amplitudes more closely represent the motion associated with the heart than Doppler blood or tissue velocity along a scan line. The amplitudes are associated with a single pixel or point or a larger region. For example, an average amplitude of the motion for each segment is determined. The motion for each spatial location for a given segment is determined and averaged. Alternatively, the motion is determined by matching a segment in one image to a segment in a subsequent image. A single motion vector is determined for the entire segment.

The amplitude of the motion is determined for desired time periods. The ultrasound data is associated with particular phases of the heart cycle. For example, ECG signals monitored during acquisition of the ultrasound data indicate relative phases of the ultrasound data. As another example, the tracked motion throughout a heart cycle is analyzed to identify the heart cycle and relative phase information for each image. Motion amplitude associated with desired phases is selected, such as identifying motion for a rapid filling period (e-wave), an atrial contraction (a-wave) or both the rapid filling period and the atrial contraction. An average, maximum or other parameter of the amplitude of the motion during a selected phase is extracted.

The memory 14 is a computer readable storage media. Computer readable storage media include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 14 stores the ultrasound data for or during processing by the processor 12. For example, the ultrasound data is a sequence of B-mode images or pre-detected data representing a myocardium at different times. The sequences are in a clip stored in a CINE loop, DIACOM images or other format. The ultrasound data is input to the processor 12 or the memory 14.

Other sources of medical information may be stored on the memory 14. The processor 12 classifies the heart or myocardium based on the ultrasound data analysis described herein with or without other medical data. For example, one or more different types of medical images are input from MRI, nuclear medicine, x-ray, computer themography, angiography, and/or other now known or later developed imaging modeality. Additionally or alternatively, non-image medical data is input, such as clinical data collected over the course of a patient's treatment, patient history, family history, demographic information, genetic information, billing code information, symptoms, age, or other indicators of likelihood related to the abnormality detection being performed. For example, whether a patient smokes, is diabetic, is male, has a history of cardiac problems, has high cholesterol, has high HDL, has a high systolic blood pressure or is old may indicate a likelihood of cardiac wall motion abnormality.

The display 16 is a CRT, monitor, flat panel, LCD, projector, printer or other now known or later developed display device for outputting determined information, such as displaying amplitude of motion information. For example, the processor 12 causes the display 16 at a local or remote location to output data indicating an amplitude or other process related information with or without images. The output may be stored with or separate from any medical data.

Different types of displays may be provided. For example, the amplitudes for each of a plurality of segments of the heart are displayed. A bulls-eye (see FIG. 3) displays the amplitude for each of the plurality of segments substantially simultaneously. Numerical or color coded displays may be used. As another example, the portions of a B-mode image representing the myocardium or other region of interest are color coded as a function of the amplitude. As another example, an amplitude of motion at a user selected region or point is displayed. As the user selects different locations, values representing the amplitude are displayed sequentially. As yet another example, whether the motion is normal or abnormal is displayed for each of a plurality of locations. The abnormal and normal characterization is based on the amplitude information alone or includes other information for classification. A binary display or overlay is provided, but a range of three or more classes of normal and abnormal may be used.

A computer readable storage medium has stored therein data representing instructions executable by a programmed processor for automated analysis of heart function with ultrasound. The automatic or semiautomatic operations discussed above are implemented, at least in part, by the instructions. In one embodiment, the instructions are stored on a removable media drive for reading by a medical diagnostic imaging system or a workstation networked with imaging systems. An imaging system or work station uploads the instructions. In another embodiment, the instructions are stored in a remote location for transfer through a computer network or over telephone lines to the imaging system or workstation. In yet other embodiments, the instructions are stored within the imaging system on a hard drive, random access memory, cache memory, buffer, removable media or other device.

The memory 14 is operable to store instructions executable by the programmed processor 12. The instructions are for automated analysis of heart function with ultrasound. The functions, acts or tasks illustrated in the figures or described herein are performed by the programmed processor 12 executing the instructions stored in the memory 14 or a different memory. For example, the instructions provide for acquiring ultrasound data representing a heart at different times by receiving from memory a sequence of B-mode images in a clip, such as a clip associated with scanning from a same transducer for each of the B-mode images and associated with receiving along a plurality of scan lines substantially simultaneously. The programmed processor 12 automatically determines two or three dimensional motion for each of a plurality of segments of the heart from the ultrasound data with a trained classifier or other programmed technique. Amplitudes of each of the two or three dimensional motions are identified during an e-wave period, an a-wave period or both the e-wave and a-wave periods. For example, a maximum amplitude is extracted based on the instructions for each of the plurality of segments.

The functions, acts or tasks are independent of the particular type of instructions set, storage media, processor or processing strategy and may be performed by software, hardware, integrated circuits, film-ware, micro-code and the like, operating alone or in combination. The instructions are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like.

FIG. 2 shows one embodiment of a method for automated analysis of heart function with ultrasound. The method is implemented using the system 10 of FIG. 1 or a different system. Additional, different or fewer acts than shown in FIG. 2 may be provided in the same or different order. For example, act 26 is not performed, but instead the amplitude data is stored, provided by audio or transmitted. The acts 22 and 24 are performed automatically, such as after triggering by user selection. A processor implements the acts 22 and 24 without user selection or interference. Alternatively, a user assists in refining the motion or amplitude information, such as by altering computer generated myocardial wall borders, by tracing the borders or by identifying e-wave and/or a-wave time periods associated with images.

In act 20, ultrasound data representing a heart at different times is acquired. B-mode data, pre-detected data or both B-mode and pre-detected data are acquired. The data is acquired by scanning one or more regions of a patient with acoustic energy. In response to echoes, the data is formed by an ultrasound imaging system. The scanning uses single transmit and receive beams. Alternatively, the scanning receives multiple receive beams along a respective plurality of scan lines substantially simultaneously in response to a single transmit event. For example, 2-4 receive beams are received in response to a single focused or converging transmit beam. As another example, receive beams or data for an entire region are formed in response to a single plane or diverging wave transmit. These multi-beam techniques may significantly increase the speed at which ultrasound images are acquired. Other now known or later developed scanning techniques or formats may be used.

The scanned region is the heart of a patient. For example, one or more acoustic windows through the ribs of a patient are used to acquire two dimensional images of a patient. As another example, a transesophageal probe is used to acquire two or three dimensional sets of data. As yet another example, an intravenous catheter is used to acquire two or three dimensional sets of data.

The ultrasound data is acquired in real time with the scanning. Alternatively, the data is acquired from a memory or other storage shortly or a long time after scanning. Whether real-time or later acquired, the digital ultrasound data is provided for extracting motion information for the heart.

In act 22, two or three dimensional motion for a plurality of locations is determined with a processor from the ultrasound data. The two or three dimensional motion represents the motion or velocity of the locations without reliance only on motion to and from a transducer. The motion is either along scan lines or at a non-zero angle to scan lines. A maximum velocity vector for each location is determined from B-mode or pre-detected data. By tracking form B-mode data or pre-detected ultrasound data, the true motion of the myocardium is measured, rather than just the motion along the ultrasound beam. Data representing a volume allows identification of motion in three dimensions. Data representing an area allows identification of motion in two dimensions. By using B-mode data, the motion of the myocardium is substantially simultaneously captured for each segment of the heart in a given view.

The motion of the myocardium is automatically detected and tracked in 2D imaging or 3D imaging. For example, a trained classifier automatically determines the motion as disclosed in U.S. Pat. No. ______ (Published Application No. 2004/0208341), the disclosure of which is incorporated herein by reference. As another example, detection and tracking is performed using correlation-based processing of pre-detected ultrasound data. Other now known or later developed processes for detecting and/or tracking heart tissue may be used, such as border tracing, thresholding, filtering, speckle tracking, minimum sum of absolute differences tracking of regions, or feature tracking.

The motion is determined in act 22 for different locations. For example, the myocardium is segmented into 16 segments, but a fewer or greater number of segments may be used. Standardized or user selected segments are provided. The motion for a given segment is the average motion for the region, but the motion at the center of the region or other criteria may be used. In alternative embodiments, the different locations are each a point. In yet other alternative embodiments, the two or three dimensional motion for a single location (e.g., point or region) is determined.

In act 24, an amplitude (e.g., velocity magnitude) of the two or three dimensional motion is extracted. A processor automatically extracts the amplitude for each location, such as determining the amplitude for each segment of a myocardial wall. The amplitude is the magnitude of the motion. The amplitude is for a given time or over a time period. For example, the maximum or average amplitudes during a rapid filling period, an atrial contraction or both the rapid filling period and the atrial contraction phases of the heart are determined. Other time periods with or without overlap may be used.

In act 26, information is displayed as a function of the extracted amplitude or amplitudes. For example, an e-wave, a-wave or both e-wave and a-wave information for a single heart cycle or averaged over multiple heart cycles is displayed for each location. As another example, a ratio (e/a) of the amplitudes during the rapid filling period and the atrial contraction is calculated. Diastolic function is analyzed, in part, from the ratio e/a. A waveform representing the amplitude information as a function of time throughout a desired phase or across multiple heart cycles may be displayed.

FIG. 3 shows one embodiment for displaying the amplitude for each of a plurality of segments of the heart. Sixteen segments are shown relative to the heart in a bulls-eye. The ratio of e-wave and a-wave amplitudes is provided as numerical values for each of the plurality of segments substantially simultaneously. In alternative embodiments, the e-wave and/or a-wave amplitudes are numerically displayed with or without the ratio values.

Values representing the amplitude (e.g., ratio values) at user selected regions may also or alternatively be displayed sequentially. For example, a video clip of ultrasound images is played. The user selects a region of the heart, such as by placing a cursor over the region or other selection. The system responds with a velocity curve showing amplitude as a function of time, extracted values for e and a, and/or the ratio e/a. The selected region may or may not correspond to a segment region defined by the ASE. Other regions may be subsequently selected.

In another embodiment of displaying, the amplitude information is used to determine whether a location or segment operates normally or abnormally. Using classification, thresholds, filters or other process, normal or abnormal operation of the locations is identified as a function of the respective amplitudes. For example, whether the diastolic phase is normal or abnormal is determined for each segment based on the maximum amplitude of motion. Other heart or medical parameters may be used for classification as normal or abnormal. For example, amplitude of motion information is combined with volume and filling rates of the heart to determine abnormal or normal operation. An image is color coded based on the determination, or textual indication is output.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for automated analysis of heart function with ultrasound, the method comprising:
acquiring ultrasound data representing a heart at different times;
determining with a processor two or three dimensional motion for a plurality of locations from the ultrasound data; and
extracting an amplitude of the two or three dimensional motion during a rapid filling period, an atrial contraction or both the rapid filling period and the atrial contraction.

2. The method of claim 1 wherein acquiring ultrasound data comprises acquiring B-mode data, pre-detected data or both B-mode and pre-detected data.

3. The method of claim 1 wherein acquiring comprises acquiring along a plurality of scan lines substantially simultaneously.

4. The method of claim 1 wherein determining comprises determining with a trained classifier.

5. The method of claim 1 wherein determining comprises determining correlating pre-detected ultrasound data.

6. The method of claim 1 wherein determining two or three dimensional motion comprises determining a maximum velocity vector other than along scan lines.

7. The method of claim 1 wherein determining for a plurality of locations comprises determining two or three dimensional motion for each of a plurality of segments on the heart.

8. The method of claim 1 wherein extracting the amplitude comprises extracting with the processor a maximum amplitude for each of the plurality of locations.

9. The method of claim 8 wherein determining and extracting with the processor comprise automatic determining and extracting.

10. The method of claim 1 further comprising calculating a ratio of the amplitude during the rapid filling period and the atrial contraction.

11. The method of claim 1 further comprising displaying the amplitude for each of a plurality of segments of the heart.

12. The method of claim 11 wherein displaying comprises displaying a bulls-eye with the amplitude for each of the plurality of segments displayed substantially simultaneously or displaying values representing the amplitude at user selected regions sequentially.

13. The method of claim 1 further comprising identifying normal or abnormal operation of the plurality of locations as a function of the respective amplitudes and at least one other heart parameter.

14. A computer readable storage medium having stored therein data representing instructions executable by a programmed processor for automated analysis of heart function with ultrasound, the storage medium comprising instructions for:
acquiring ultrasound data representing a heart at different times;
determining two or three dimensional motion for each of a plurality of segments of the heart from the ultrasound data; and
identifying amplitudes of each of the two or three dimensional motions during an e-wave period, an a-wave period or both the e-wave and a-wave periods.

15. The instructions of claim 14 wherein acquiring ultrasound data comprises receiving from memory a sequence of B-mode images in a clip associated with scanning from a same transducer for each of the B-mode images and associated with receiving along a plurality of scan lines substantially simultaneously.

16. The instructions of claim 14 wherein determining comprises determining with a trained classifier.

17. The instructions of claim 14 wherein identifying the amplitude comprises extracting a maximum amplitude for each of the plurality of segments.

18. A system for automated analysis of heart function with ultrasound, the system comprising:
a memory operable to store ultrasound data representing a myocardium at different times;
a processor operable to determine multi-dimensional motion for each of a plurality of locations from the ultrasound data, and operable to determine amplitudes of the multi-dimensional motions of the locations during a rapid filling period, an atrial contraction or both the rapid filling period and the atrial contraction; and
a display operable to display information as a function of the amplitudes.

19. The system of claim 18 wherein the ultrasound data comprises a sequence of B-mode images in a clip.

20. The system of claim 18 wherein the processor comprises a classifier.

21. The system of claim 18 wherein the locations are myocardial wall segments, and the processor is operable to extract a maximum amplitude for each of the segments.

22. The system of claim 18 wherein the information is the amplitudes for each of a plurality of segments of the heart.

23. The system of claim 18 wherein the information is a bulls-eye with the amplitude for each of the plurality of segments displayed substantially simultaneously or values representing the amplitude at user selected regions sequentially.

24. The system of claim 18 wherein the information is an indication of normal or abnormal operation of the plurality of locations.
